(19) United States
(12) Patent Application Publication
(10) Pub. No.: US 2007/0233154 A1
(43) Pub. Date: Oct. 4, 2007

(54) TOOL FOR REMOVING BROKEN INTRAMEDULLARY RODS

(76) Inventors: Shreedhar Kale, Bensalem, PA (US); Matthew C. Klimash, Piscataway, NJ (US); Glenn A. Rupp, Highland Lakes, NJ (US)

Correspondence Address: ARTHUR L. LESSLER, 540 OLD BRIDGE TURNPIKE, SOUTH RIVER, NJ, 08882, US

(21) Appl. No.: 11/672,621

(22) Filed: Feb. 8, 2007

Related U.S. Application Data
(60) Provisional application No. 60/774,304, filed on Feb. 17, 2006.

(51) Int. Cl. A61F 2/00

(52) U.S. Cl. 606/99

(57) ABSTRACT

A broken intramedullary rod removal tool for extracting the distal end of a broken intramedullary rod from bone in which it is embedded. In a first embodiment the tool has an extraction shaft, an expansion rod disposed and longitudinally movable within the shaft, a strike plate adapter secured to the proximal end of the extraction shaft, a thumb screw secured to the strike plate adapter, and a strike plate secured to the strike plate adapter. The distal end of the extraction shaft is inwardly tapered and has a split-bulb tip, so that the expansion shaft distal end can be flared out by insertion of the distal end of the expansion shaft therein, to expand the tip so that it engages the distal end of the broken intramedullary rod, thus permitting the distal portion of the broken rod to be removed by striking the strike plate to force out the expansion shaft and tip-engaged broken rod end. In a second embodiment the split-bulb tip in its relaxed position is outwardly flared to engage the distal end of the broken intramedullary rod and is initially inwardly deflected by a controlling cap to permit the tip to be moved distally within a casing to a position where the tip can engage the broken end of the rod when the controlling cap is moved to allow the tip to flare out.

### CROSS-REFERENCE TO RELATED APPLICATION

This application claims the benefit of U.S. Provisional Application Ser. No. 60/774,304 filed Feb. 17, 2006 and entitled Tool for Removing Broken Intramedullary Rods.

### BACKGROUND OF THE INVENTION

The present invention relates to a tool for removing an intramedullary rod, or portion thereof, which is embedded in bone or other hard tissue.

Intramedullary rods are routinely used to reduce and stabilize fractures in long bones such as the femur and tibia. They are typically inserted into reamed bone marrow canals and secured in place with bone screws. Bone screw holes are typically clustered in two groups along the length of the intramedullary rod. One group of holes is concentrated toward the distal end of the rod while the other group of holes is concentrated toward the proximal end thereof. Such screw holes typically have their center axes oriented in the lateral-medial direction at one end of the rod and in the anterior-posterior direction at the opposite end.

Intramedullary rods are typically manufactured with a cannular shaft having a central longitudinal hole with the proximal end thereof internally threaded. The thread size is often unique to a given manufacturer. Intact intramedullary rods are typically removed using tools such as the Winquist III Universal Extraction System developed and manufactured by S.S. White Medical Products Inc., Piscataway, N.J. That extraction system employs one of a number of different size threaded adapters to releasably engage the threads in the hole at the proximal end of the intramedullary rod to be removed. Once the adapter is engaged and subsequently attached to a strike plate, a mallet is repeatedly struck against the strike plate to apply force in a proximal direction so as to forcibly dislodge the intramedullary rod from the intramedullary canal of the bone. A slap hammer may also be used for this purpose.

Discontinuities in the slender cannular intramedullary rod, such as those at the bone screw holes, are often sites of intramedullary rod fractures. As a result, distal rod segments often remain deep within the intramedullary canal after the proximal segment of the intramedullary rod is removed.

A device which is typically used to remove the broken distal end of an intramedullary rod comprises three or more ball-tipped guide wires which are successively snaked down through the intramedullary canal and through the cannula of the broken intramedullary rod segment until each of the ball tips extends beyond the distal end of the broken intramedullary rod segment. Once all of the ball tips are in place, the proximal ends of the guide wires are pulled. The cluster of ball tips then presents as a larger bulbous mass which prevents the tips from being withdrawn through the cannula of the intramedullary rod segment, so that the ball tip cluster engages the distal end of the broken rod segment and forces it outward. Increasing force is applied until the broken intramedullary rod segment is dislodged from the intramedullary canal of the bone.

This prior art extraction method, however, is a relatively slow process which is somewhat cumbersome to carry out.

Accordingly, an object of the present invention is to provide a tool capable of removing the distal end of a broken intramedullary rod relatively quickly and easily.

### SUMMARY OF THE INVENTION

A tool for removing broken embedded intramedullary rods has a tube with a proximal end and a distal end. The distal end of the tube has a resilient split tip with at least two prongs. Within the tube is a prong control mechanism which causes or allows the prongs to move between a first position where the prongs may move axially entirely within the tube and a second position wherein the prongs are outwardly flared and adapted to engage a distal part of a broken intramedullary rod. An operating knob or the like is coupled to the proximal end of the tube for operating the prong control mechanism.

### IN THE DRAWING

FIG. 1A is an isometric view of a broken intramedullary rod removal tool according to a first embodiment of the present invention.

FIG. 1B is an isometric view of the distal tip and expansion rod of the tool shown in FIG. 1A with the expansion rod extended.

FIG. 2A is an exploded isometric view of the tool shown in FIG. 1A.

FIG. 2B is a left side view of the tool shown in FIG. 1A.

FIG. 2C is a cross-sectional view of said tool taken along the cutting plane A—A shown in FIG. 2B, with the enlarged distal end of the extraction shaft in engagement with the distal end of the distal portion of a broken intramedullary rod to be removed.

FIG. 3A is an isometric view of the distal tip and expansion rod of the tool shown in FIG. 1A with the expansion rod retracted.

FIG. 3B is a right side elevation view of the proximal end of the extraction shaft of the tool shown in FIG. 1A.

FIG. 4 is an isometric view of the strike plate adapter of the tool shown in FIG. 1A.

FIG. 5 is an isometric view of the thumb screw portion of the tool shown in FIG. 1A

FIG. 6 is a left side cross-sectional view of the distal portion of the tool shown in FIG. 2C, disposed within the distal portion of a broken intramedullary rod to be removed, and with the expansion rod retracted.

FIG. 7 is a left side cross-sectional view of the proximal portion of the tool shown in FIG. 2C.

FIG. 8 is a left side cross-sectional view of the distal portion of the tool shown in FIG. 2C, disposed within the distal portion of a broken intramedullary rod to be removed, and with the expansion rod extended.

FIG. 9 is a left side cross-sectional view of a broken intramedullary rod removal tool according to a second embodiment of the present invention, disposed within the distal portion of a broken intramedullary rod to be removed, and with the tool's controlling cap urged toward the proximal end of the tool.

FIG. 10 is a left side cross-sectional view of the tool shown in FIG. 9, with the tool's controlling cap relaxed.

### DETAILED DESCRIPTION

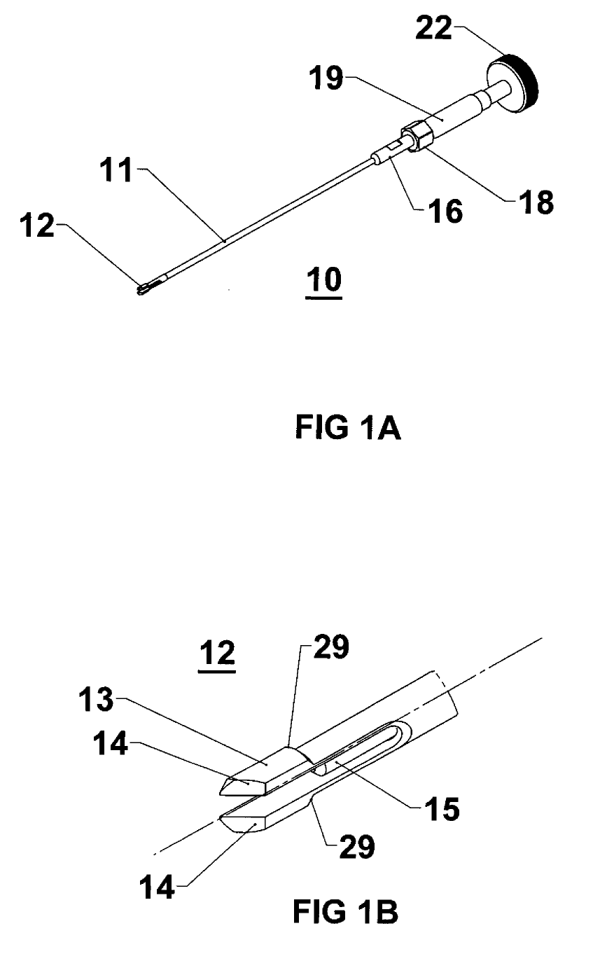

As shown in FIGS. 1A and 1B, a broken intramedullary rod removal tool 10 has an elongated cannular or tubular extraction shaft 11 with a distal end 12 comprising a resilient split-bulb tip 13 having lead-in edges 14 which are partially beveled, preferably at about a 45° angle, which tip is capable of being resiliently flared open. Each prong of the tip 13 has a shoulder 29 at the proximal end of the tip.

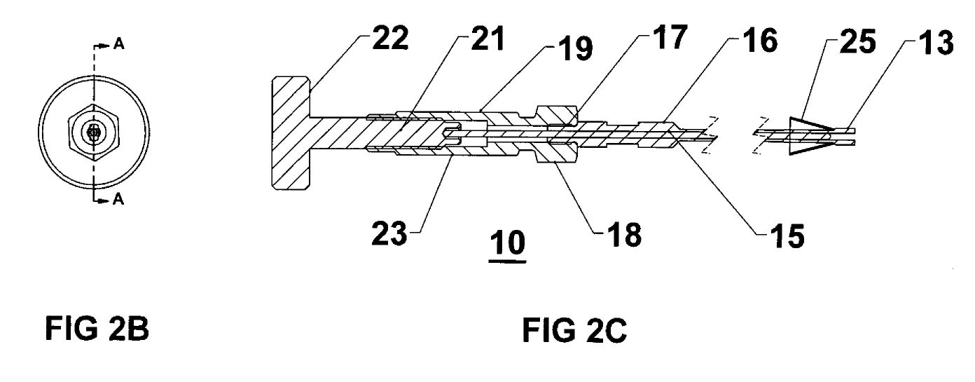

As shown in FIG. 3B, the proximal end of the extraction shaft 11 comprises an enlarged portion 16 with an externally threaded end part 17.

The end part 17 is disposed in and threadably engaged with the hexagonally shaped internally threaded distal end 18 of a strike plate adapter 19.

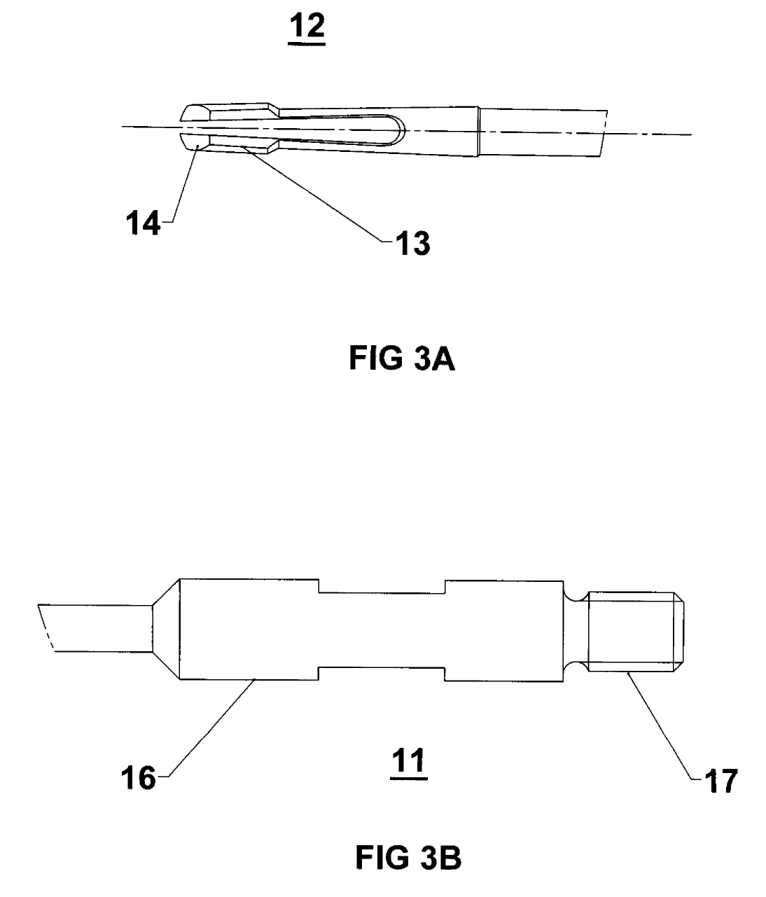

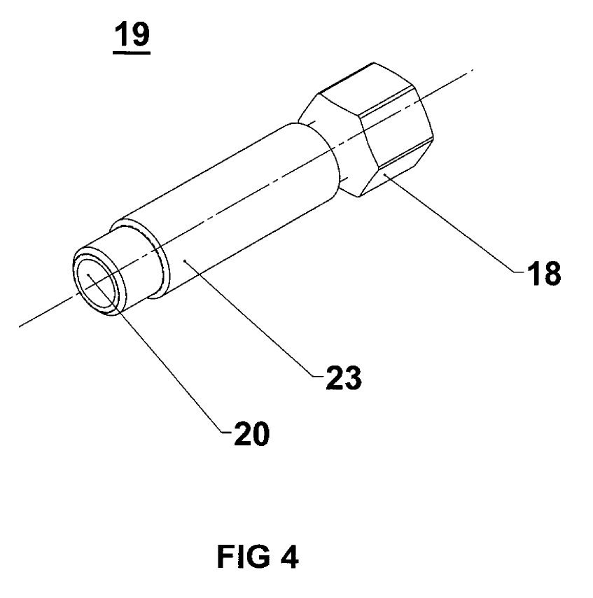

As best seen in FIG. 4, the strike plate adapter 19 has an internally threaded proximal end 20 for receiving the externally threaded distal end 21 of a thumb screw 22, and a major central portion 23 which is externally threaded to be engaged by a C-frame of conventional design (not shown). A suitable C-frame is currently sold by Shukla Medical Inc., 151 Old New Brunswick Road, Piscataway, N.J. 08854 as part of its Winquist I™ Universal Extraction System. The thumb screw 22 has a knurled rotatable knob 24 at its proximal end.

An expansion rod 15 is disposed and longitudinally movable within the extraction shaft 11.

The split bulb tip 13, when not in contact with the expansion rod 15, has the adjacent portions thereof slightly tapered toward each other, as best seen in FIG. 3A. The tip is intentionally deformed symmetrically inward toward the center axis of the shaft and is adapted to be resiliently outwardly deflected by the distal end of the expansion rod 15 when the rod is urged toward said tip.

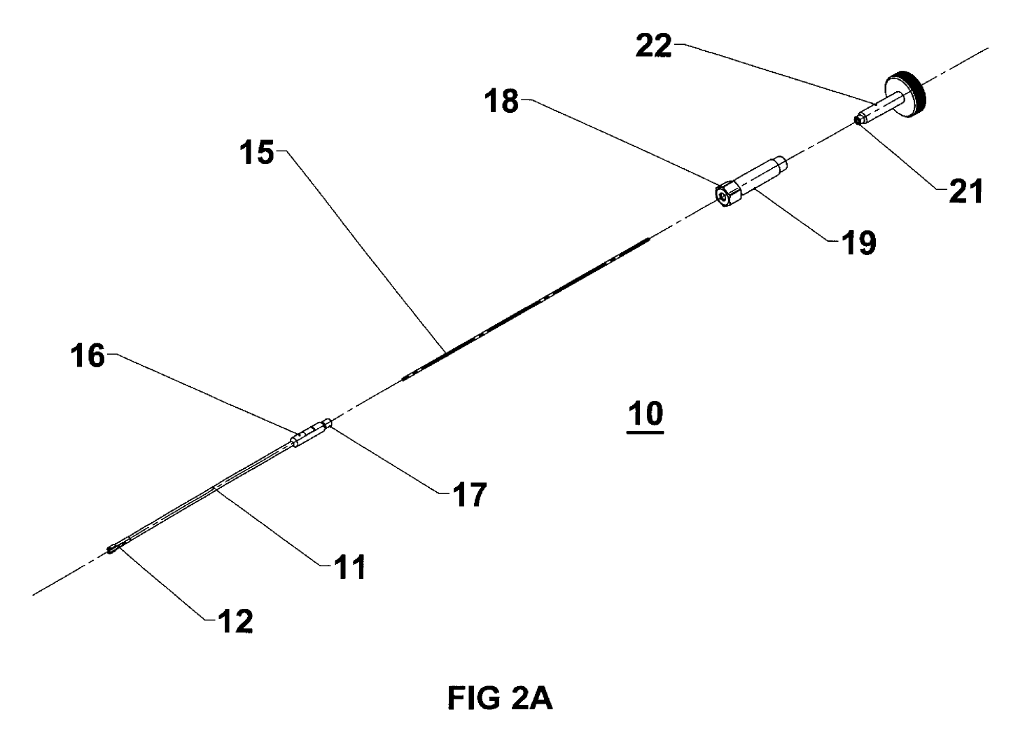

The distal ends of intramedullary rods usually have a conical shape, a typical intramedullary rod distal end segment 25 being shown in FIGS. 2C, 6, 8, 9 and 10.

It is preferable to provide a kit which includes a tool according to the invention, with extraction shafts of various lengths and various thread configurations, so that the tool can be used to remove intramedullary rods of various manufacturers as well as broken distal ends thereof.

The purpose of tapering the distal tip with a lead-in angle of about 45° is to prevent stubbing upon entry of the tip into the broken intramedullary rod segment 25.

The expansion rod 15 has an elongated cylindrical shape and is preferably a solid shaft of uniform diameter, adapted to fit within the longitudinal axial hole of the extraction shaft 11 so as to outwardly deflect the tip 13 at the end of the extraction shaft when the expansion rod is moved toward the tip 13.

The thumb screw 22 is threadably engaged with the strike plate adapter 19 in such a way that turning the thumb screw in a given direction forces the expansion rod 15 distally, to engage and flare out the extraction shaft tip 13.

To use the tool, the expansion rod 15 is moved distally within the extraction shaft 11 until it bottoms out on the distal bulb tip 13. This subassembly is then threaded into the strike plate adapter 19 and attached to the strike plate (not shown) via a hex nut (not shown) threaded onto the central portion 23 of the strike plate adapter 19.

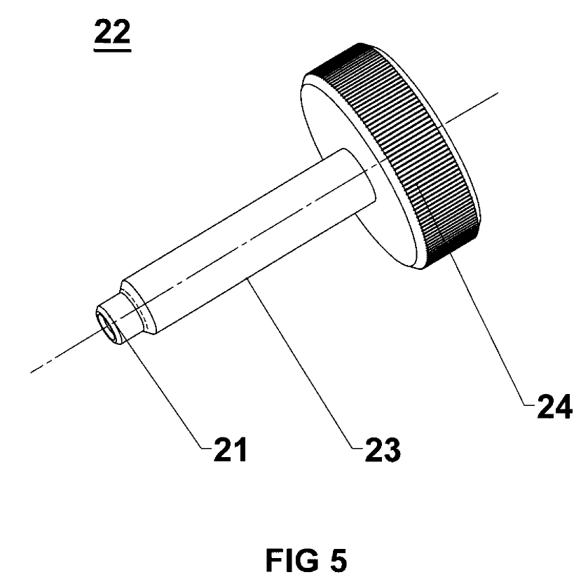

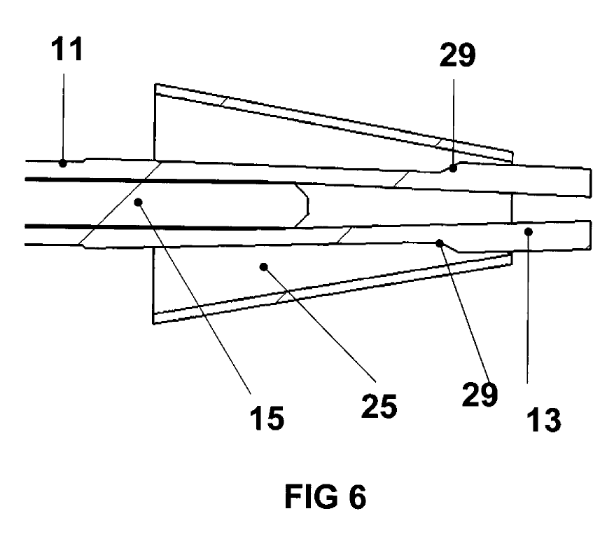

This assembly is then fed down through the intramedullary rod segment 25 until the tip 13 of the extraction shaft 11 extends beyond the distal tip of the broken intramedullary rod segment 2—see FIG. 6 which shows the tip in an intermediate position in the process of being so extended.

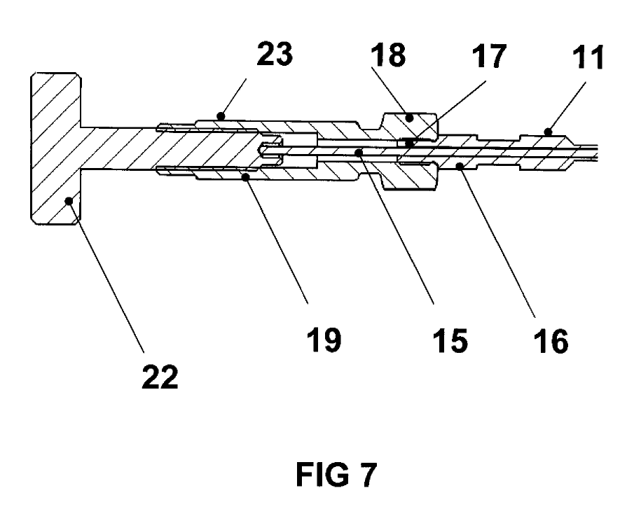

The thumb screw 22 is then threaded into the proximal end 20 of the strike plate adapter 19 and rotated to advance the expansion shaft 15 forward, i.e. distally. This action causes the expansion rod to engage the distal end 12 of the extraction rod 11 and cause the tip 13 to flare out to a diameter greater than the cannula diameter of the intramedullary rod so that the extraction rod tip shoulders 29 can engage adjacent portions of the intramedullary rod segment, as shown in FIGS. 2C and 8.

A mallet (not shown) is used to repeatedly strike the strike plate (not shown) in a proximal direction so as to forcibly dislodge the broken intramedullary rod segment 25 from the intramedullary canal of the bone in which it is embedded. Alternatively, a slap hammer arrangement of conventional type may be used for this purpose.

The procedure is then reversed to release the removed intramedullary rod segment from the extraction shaft. That is, the thumb screw 22 is rotated in a direction opposite to the given direction and the extraction rod is at least partially withdrawn (moved proximally) from the interior of the extraction shaft 11, allowing the resilient tip 13 to no longer flare out, so that the broken intramedullary rod segment can then be removed from the extraction shaft.

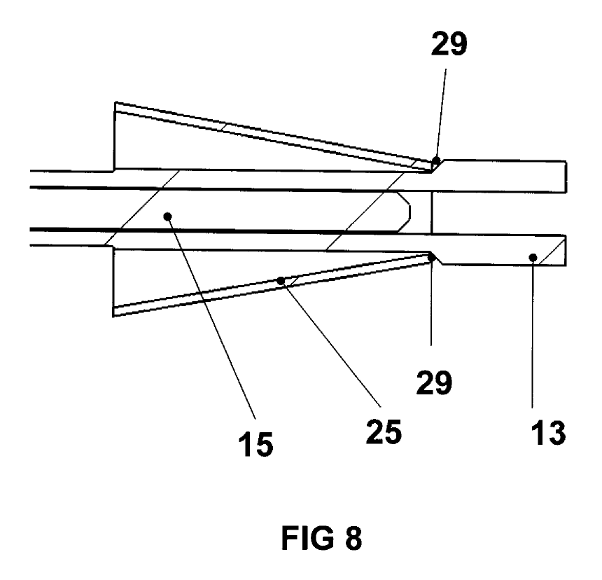

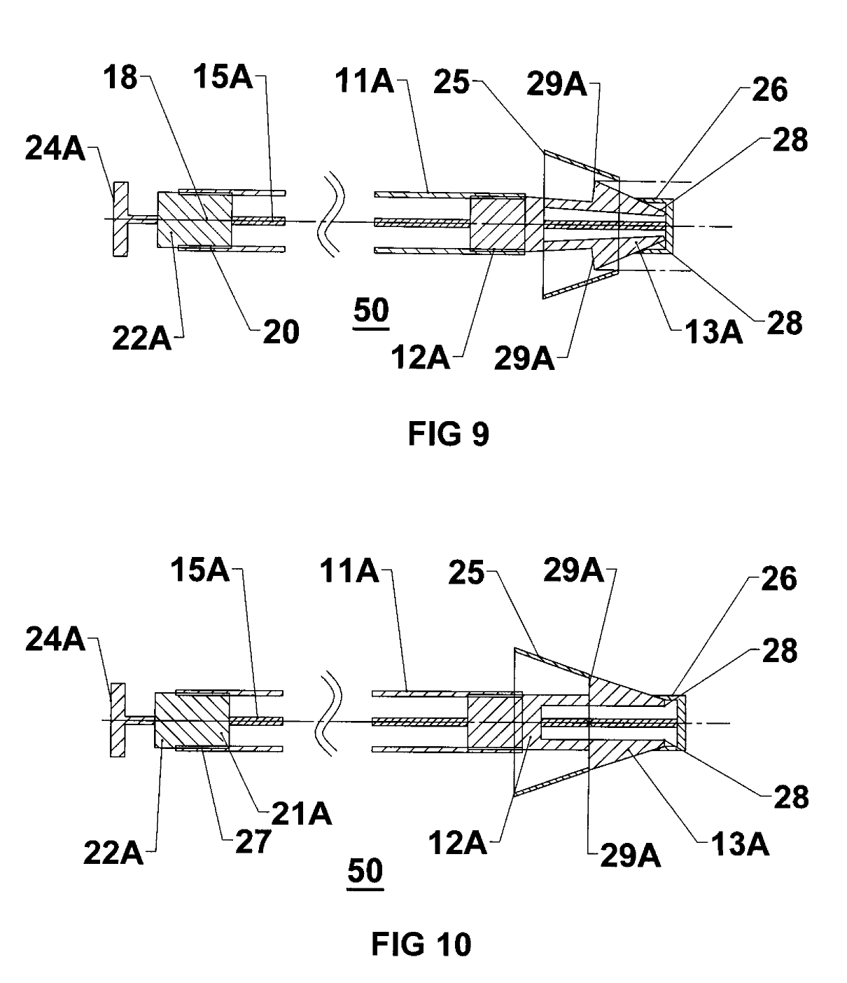

In the second embodiment shown in FIGS. 9 and 10, an arrangement is employed wherein a resilient split tip is flared out in its relaxed position and is caused to flare in to permit insertion of the tip into a broken intramedullary rod to be removed. That is, in the first embodiment the tip in its unstressed position is flared in (or not flared out) and is caused to be flared out by an expansion rod; whereas in the second embodiment the tip in its unstressed position is flared out and is caused to be flared in (or no longer flared out) by a controlling cap as discussed below.

The tool 50 shown in FIGS. 9 and 10 comprises an elongated tubular casing 11A with a distal member 12A secured thereto, said distal member having a slotted tip 13A.

A connecting shaft 15A is disposed within the casing. A controlling cap 26 is affixed to the distal end of the shaft 15A and a thumb screw 22A is affixed to the proximal end of the shaft. The thumb screw 22A has an externally threaded proximal portion 21A which is threadably engaged with an internally threaded proximal end portion 27 of the casing 11A. The casing end portion 27 is also externally threaded.

To begin the process for removing the distal end of the broken intramedullary rod segment 25, as shown in FIG. 9 the thumb screw knob 24A is rotated to apply proximal force to the connecting shaft 15A and attached controlling cap 26 to pull the cap toward the thumb screw 22A so that the cap forces or urges the prongs 28 of the tip 13A to move toward each other and closer to the axis of the casing 11A. This movement of the tip prongs 28 reduces the overall width of the slotted tip to a dimension smaller than the diameter of the smallest section of the intramedullary rod segment 25 so that the tip can be extended through the distal end of the rod segment.

The tool is then inserted into the intramedullary canal until the widest portion of the slotted tip 13A passes the distal end of the intramedullary rod segment 25. The knob 24A of the thumb screw 22A is now rotated in a direction to relieve proximal pressure on the controlling cap 26 which in turn releases the pressure exerted by the controlling cap 26 on the slotted tip 13A, allowing the prongs 28 to flare out or open, as shown in FIG. 10.

In this flared out or open position, the widest section of the slotted tip 13A is wider than the internal diameter of the smallest section of the intramedullary rod segment 25, so that when the tool 50 is urged proximally the shoulders 29A of the flared out or open tip 13A engage an adjacent part of the rod segment 25 to urge the rod segment proximally so as to withdraw it from the bone in which it is embedded.

To urge the tool 50 proximally, a strike plate or slap hammer may be threadably coupled to the externally threaded portion 21A of the casing 11A, and the strike plate then struck with a mallet or the slap hammer operated for this purpose.

A suitable slap hammer-like arrangement is currently sold by Shukla Medical Inc., 151 Old New Brunswick Road, Piscataway, N.J. 08854 as part of its Winquist II™ Universal Extraction System.

In the event extraction of the intramedullary rod segment 25 needs to be aborted, the thumb screw knob 24A is rotated to urge the controlling cap 26 proximally so as to cause the prongs 28 to collapse, i.e. move toward the axis of the casing 11A again; and the tool 50 can then be moved proximally to remove the casing and associated tool components from the intramedullary canal without leaving any foreign objects in the canal.

I claim:

1. A tool for removing broken embedded intramedullary rods, comprising:
a tube having a proximal end and a distal end, the distal end of the tube having a resilient split tip with at least two prongs;
prong control means disposed within the tube for causing said prongs to move between a first position where the prongs may move axially entirely within the tube and a second position wherein the prongs are adapted to engage a distal part of a broken intramedullary rod; and
means coupled to the proximal end of said tube for operating said prong control means.

2. The tool according to claim 1, wherein said prongs have shoulders for engaging said distal part of said broken intramedullary rod.

3. The tool according to claim 2, wherein said control means comprises a thumb screw.

4. The tool according to claim 3, wherein said prong control means comprises an expansion rod.

5. The tool according to claim 3, wherein said prong control means comprises a controlling cap contiguous with the prongs.

6. A tool for removing broken embedded intramedullary rods, comprising:
a cannular extraction shaft having a proximal end and a distal end,
the distal end of the extraction shaft having a resilient split tip with at least two prongs;
an elongated expansion rod disposed and longitudinally movable within the extraction shaft, said rod having a distal end adjacent said tip and a proximal end,
said rod and tip being dimensioned and positioned so that movement of the expansion road toward the tip causes the prongs to be deflected away from each other and radially outward from the axis of the extraction rod;
so that the prongs may move axially entirely within the extraction rod when the expansion rod is a given distance away from the tip and may extend outward from the extraction rod to engage a distal part of the broken rod when the expansion rod is within a predetermined distance of the tip.

7. The tool according to claim 6, further comprising a strike plate adapter having a distal end secured to the proximal end of the extraction shaft, and a threaded proximal end.

8. The tool according to claim 7, further comprising a rotatable member threadably engaged with the strike plate adapter and having a distal end portion adjacent the proximal end of the expansion rod.

9. The tool according to claim 8, further comprising means for connecting a driving device to said strike plate adapter to urge said tool proximally.

10. A tool for removing broken embedded intramedullary rods, comprising:
a cannular casing having a proximal end and a distal end, the distal end of the casing having a resilient split tip with at least two prongs;
elongated connecting means disposed within the casing, said connecting means having a distal end adjacent said tip and a proximal end;
a controlling cap secured to the distal end of the connecting shaft and adapted to engage said prongs so as to urge them together when the cap is moved toward the proximate end of the casing and to all them to move away from each other and radially outward from the axis of the casing when the cap is allowed to move away from the proximate end of the casing; and
rotatable means for moving said connecting shaft axially within said casing;
so that the prongs may move axially entirely within the casing when the cap is urged toward the proximal end of the casing with a predetermined force and may extend outward from the casing to engage a distal part of the broken rod when the force urging the cap toward the proximal end of the casing is less than a predetermined amount.

11. The tool according to claim 10, wherein said rotatable means comprises a thumb screw threadably engaged with the casing and having a distal end secured to the proximal end of the connecting shaft.

12. The tool according to claim 11, wherein said elongated connecting means comprises a shaft.

13. The tool according to claim 12, wherein said rotatable means comprises a thumb screw threadably engaged with the casing and having a distal end secured to the proximal end of the connecting shaft.
